(19) United States
(12) Patent Application Publication
(10) Pub. No.: US 2016/0245821 A1
(43) Pub. Date: Aug. 25, 2016

(54) METHOD OF DEVELOPING SMALL MOLECULE PEPTIDE CONJUGATES FOR BIOMEDICAL APPLICATIONS

(71) Applicant: Proteorex Therapeutics, Toronto (CA)

(72) Inventor: Aman Iqbal, Toronto (CA)

(21) Appl. No.: 15/002,201

(22) Filed: Jan. 20, 2016

Related U.S. Application Data
(63) Continuation of application No. PCT/IB2016/050255, filed on Jan. 20, 2016.
(60) Provisional application No. 62/105,585, filed on Jan. 20, 2015.

(51) Int. Cl. G01N 33/68; G06F 19/18; B01J 19/00; C40B 30/02; G01N 21/65

(52) U.S. Cl. CPC G01N 33/6845; C40B 30/02; G01N 21/658; B01J 19/0046; G06F 19/18; B01J 2219/00725; B01J 2219/00596

(57) ABSTRACT

This application provides a platform design wherein a substrate peptide of the protein-protein interaction complex is tagged with a variety of small molecule fragments using amide chemistry. The method of hit discovery coupled with subsequent amide-mediated rapid diversification for lead generation reduces time and cost to discovery and development of target specific small molecule leads for the inhibition of protein-protein interactions.

### RELATED APPLICATION

This application is related to and claims the benefit of priority of U.S. Provisional Application 62/105,585 filed 20 Jan. 2015, and PCT/IB2016/050255 filed 20 Jan. 2016, which are both incorporated herein, by reference in their entirety.

### FIELD OF THE INVENTION

This application is related to a novel platform technology involving discovery of small molecule fragments against protein-protein/peptide drug targets resulting in identification of small molecule lead compounds using amide coupling.

### BACKGROUND

Protein-protein interaction (PPI) drug targets are notoriously hard to target due to their broad and relatively featureless binding sites. They are often termed as the ‘high hanging fruits’ or the ‘undruggable’ targets in drug discovery. Enormous efforts towards the low hanging fruits or easier to target drug targets have reached a saturation point. There is emerging interest in newer technologies that enable rapid identification of high-quality and diverse chemical starting points for PPI inhibitor development. Traditional small molecule screening techniques like high throughput screening (FITS) often identifies large and complex molecules with undesirable physicochemical properties especially in the case of PPI drug targets. These hits are usually non-specific weak binders and require extensive medicinal chemistry efforts to improve their potency and drug-like properties. Traditional fragment based drug design (FBDD) in hit discovery has been successful against easier drug targets like kinases and hydrolases. However, the FBDD approach in PPI drug discovery has been extremely challenging, as the identification of weak moderately binding small molecule fragments to broad PPI binding sites is difficult.

Smith et al (Organic Letters 2002, 4: 4041-4044) provide design of small molecule-peptide conjugates. However, this study does not design a library of such conjugates, and use the entire conjugate as a drug/small molecule, which makes it a peptidomimetic approach.

US20110118126 provides a method for screening a compound that binds to a target wherein a bait fragment with a preselected bait moiety is reacted with multiple (plurality) fragments to form small molecule ligands. Herein, a method of identifying an optimal hit molecule by rapidly screening large number of chemical molecules after binding the molecule to the target protein by sulfide bond is described.

The present invention is a novel platform technology referred herein as the ‘Smip’ Platform, which employs in silico high throughput screening of small molecule fragments against a protein-peptide/protein-protein target of interest, while attached to the interacting peptide via an amide bond. This results in the generation of a library of small molecule peptide conjugates(SMPC). The top docked SMPC hits are selected and synthesized. The SMPC hits are then screened against the target of interest using traditional biochemical assays. Following SMPC hit identification, the small molecule fragments attached to best SMPC hits is further evolved into a small molecule lead/inhibitor against the target of interest.

The high throughput screening format ensures the exploration of a wide chemical space while significantly reducing the time and cost of screening when compared to traditional fragment based screening technologies. Also the facile diversification of the initial small molecule hits by replacement of the peptide with amine-terminated fragments will reduce the burden on medicinal chemistry for the generation of lead.

### SUMMARY OF THE INVENTION

The present invention provides a novel ‘Smip’ Platform, which employs in silico high throughput screening of small molecule fragments against a protein-peptide/protein-protein target of interest, while attached to the interacting peptide via an amide bond.

In one aspect, the invention is a method of developing small molecule lead compounds comprising generating a library of small molecule-peptide conjugates (SMPCs) by in silico highthroughput screening or highthroughput coupling of small molecule fragments to a peptide.

In another aspect, the small molecule fragment is attached to the peptide via an amide bond.

In yet another aspect, the present invention provides a method of generating a library of SMPCs comprising in silico highthroughput coupling of small molecule fragments to a peptide via an amide bond.

It is also provided that the SMPCs are synthesized on a solid surface. The solid surface is any solid membrane. The solid surface is also selected from a group consisting of cellulose, gold and glass slide.

In another aspect, the SMPCs are synthesized using highthroughput peptide microarrays.

The invention also provides a method for generating a small molecule-peptide conjugate microarray wherein the microarray ranges from a minimum of two and a maximum of a billion small molecule peptide conjugates.

The method of developing small molecule lead compounds disclosed in this application comprise the steps of:
a) in silico high throughput screening of small molecule fragments against protein-peptide or protein-protein target;
b) generating a library of SMPCs by attaching the small molecule fragments to the peptide via an amide bond;
c) selecting and synthesizing the SMPC hits;
d) screening the SMPCs against a target of interest to identify the small molecule fragment hits; and
e) conversion of the small molecule fragment to small molecule lead compounds for a corresponding target.

The invention also provides a method of generating a high throughput library of SMPCs comprising synthesizing highthroughput peptide microarrays on a solid surface by coupling small molecule building blocks to the peptide. The coupling of small molecule building blocks onto the peptide is via amide coupling. Furthermore, the coupling of small molecule to the peptide is done while immobilized on a solid surface.

The coupling of SMPCs to the solid surface is by using a linker selected from the group consisting of biotin, PEG and any solid small molecule linker.

The invention further provides a composition comprising the SMPC synthesized by the method disclosed in this application.

It is envisaged that the SMPC peptide microarrays of this invention can be used for biochemical and biophysical assays.

The SMPC of the invention is attached to gold surface in Surface Plasmon Resonance.

The SMPCs of the present invention is used in generating peptide-based vaccines by attaching small molecules to target peptide using amide coupling.

The SMPCs of the present invention is used as a diagnostic tool in wide variety of diseases.

### DESCRIPTION OF THE FIGURES

FIG. 1. ‘Fishing rod bait model’ and ‘small molecule lead conversion’

FIG. 1A detailing the generation of Small Molecule Peptide Conjugates (SMPCs).

FIG. 1B showing biochemical screening, hit selection and comparison.

FIG. 1C illustrating the conversion of an SMPC hit into small molecule inhibitor via combinatorial small molecule library preparation

FIG. 2 compares the Smip platform and the traditional fragment based screening techniques highlighting the difference in time needed to get to the small molecule lead.

FIG. 3. Fluorescent Polarization screening data for SMPCs synthesized and screened against SND1

FIG. 4. Dose response curves for SMPC hits against SND1

FIG. 5. Binding of SMPC fragments to SND1 by thermal shift and ITC

FIG. 5A. Thermal Shift and ITC of Fragment 46

FIG. 5B. Thermal Shift and Isothermal calorimetry (ITC) of Fragment 48

FIG. 6. Fluorescent Polarization screening data for SMPCs synthesized and screened against WDR5-MLL1 complex

FIG. 7. Dose Response fluorescent polarization (FP) data for SMPC hit(PRX-0065) and small molecule lead compound (PRX-0345)

FIG. 8. Dose response MLL complex activity assay comparing substrate peptide, PRX-0345 and the SMPC hit (PRX-0065)

FIG. 9. Cell based response for WDR5-MLL: MLL-AF9 transformed leukemia cells in the presence of PRX-0345, Control compounds (OICR-9429, and WDR5-103) at two different concentrations (5 and 10 μM)

### DESCRIPTION OF THE INVENTION

In order to find solutions to the limitations associated with all the current routinely used small molecule screening techniques and technologies for PPI targets in the market, the applicants have developed an innovative, next generation small molecule lead generation technology for PPIs. The technology design employs two simple steps:

The concept of the invention consists of the generation of a library of SMPCs with the same peptide sequence but with a diverse set of small molecule fragments coupled on to the peptide at the N-terminus via an amide bond, and subsequently the efficient conversion of the SMPC hits into novel, patentable, drug-like, non-peptidic small molecule inhibitors for any PPI target of therapeutic interest in less than 6 months.

In one embodiment, the ‘Fishing rod bait model’ and ‘small molecule lead conversion’ described herein as step 1 and 2 provide the technology of the invention.

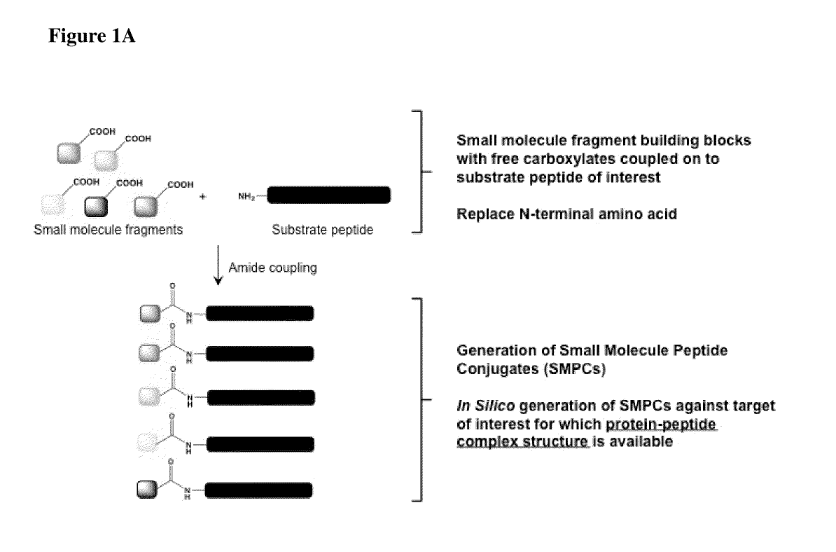

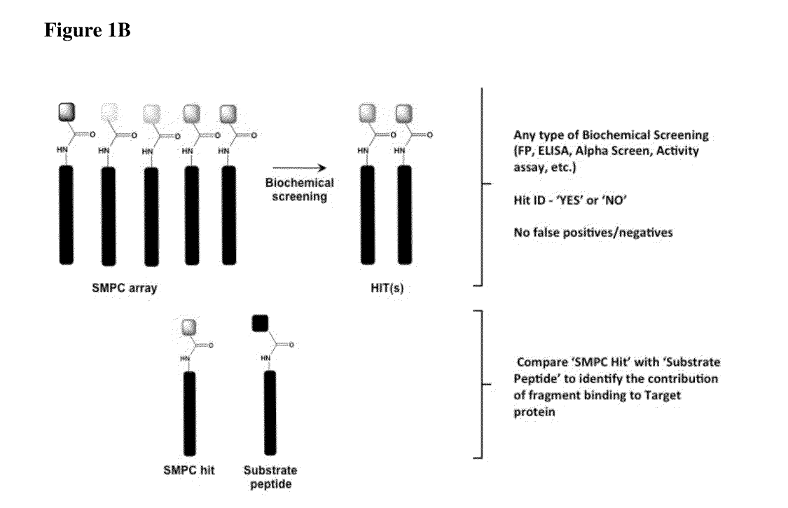

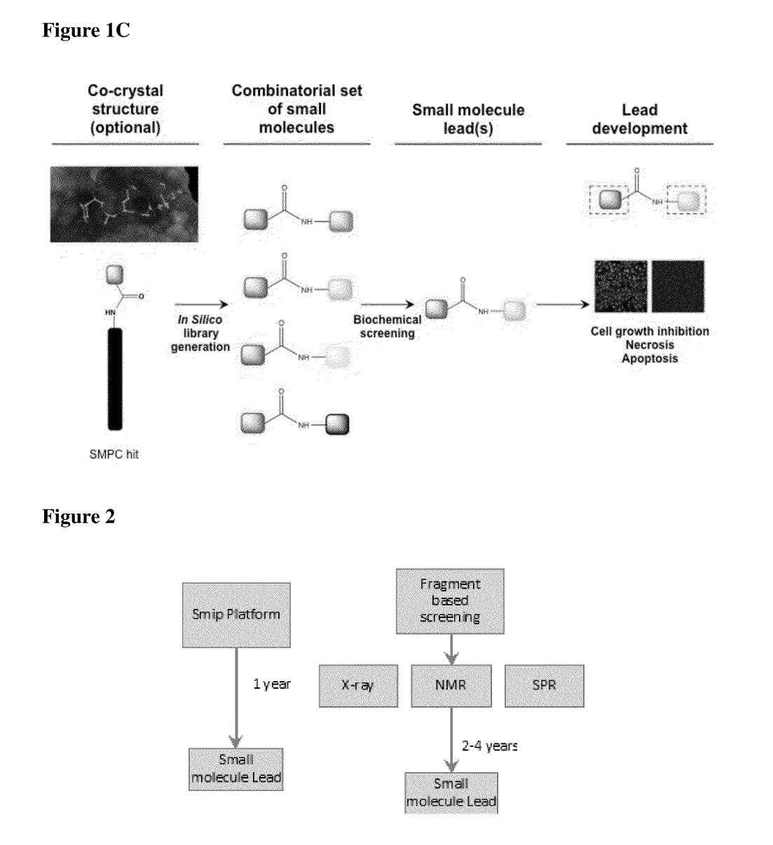

Step 1: ‘Fishing rod-bait’ model: By replacing N-terminus amino acid(s) of the peptide binder, with a diverse set of small molecule fragments while keeping the rest of the peptide constant a small molecule peptide conjugate (SMPC) (FIG. 1A) is generated. Attaching small molecule fragments onto the peptide gives an additive effect to binding and helps in guiding and anchoring the small molecule fragments into the putative binding site. If the SMPC hit shows better or similar potency when compared to the original cognate peptide, it indicates the contribution of the small molecule fragment to the binding is better than or equivalent to the amino acid it replaced (FIG. 1B).

The invention provides a method wherein the total timeline of the above step will be two months, thus significantly reducing the cost and time involved in otherwise costly and expensive screening methods.

PPI targets for which a protein-peptide crystal structure is available, the current invention provides an In Silico methodology that involves coupling of small molecule fragments onto the peptide to produce a virtual SMPC library, followed by the docking of the SMPC library to visualize the best binders based on the overall SMPC conformation, and interactions of the fragment in the binding pocket. However, for any PPI target without a protein/peptide complex structure, the inventors propose a high throughput (>1000) SMPC library generation.

Step 2: Small molecule lead conversion: Once an SMPC hit is identified, the peptide part of the SMPC hit can then be replaced by small molecule building blocks via simple combinatorial amide coupling to result in non-peptidic small molecule compounds (FIG. 1C). The non-peptidic compounds can then be screened against the target to identify the best series for development and optimization into a potent inhibitor. The latter could involve even replacing the existing amide bond with alternative handles.

It is envisaged that the total timeline for this step is 3 months.

Platform Design: The platform design of the present invention is akin to a ‘fishing rod-bait’ model. The model consists of a substrate peptide of the PPI complex tagged with a diverse set of small molecule fragments using simple amide chemistry. Using amide chemistry, the ‘hot spot’ amino acid is replaced by fragment building blocks to generate a small molecule-peptide conjugated (SMPC) library.

Applicants have also developed an In Silico methodology to generate these target specific SMPCs in addition to synthesizing them in a high throughput microarray based approach. These SMPCs can be readily synthesized using peptide microarray and also on solid surfaces like cellulose, glass and gold.

In another aspect the small molecule peptide conjugates (SMPCs) of the invention, can be attached to a gold surface on a surface plasmon resonance (SPR) chip which can then be screened against the protein of interest to identify binders. The immobilization of SMPC hits on to SPR gold chips can serve the same purpose in the identification of SMPC hits against a particular target. In this case as well the SMPC hits can be converted into small molecule lead compounds.

The SMPCs thus generated are then screened against each corresponding target in fluorescent polarization (FP) assays. By comparing the binding affinity of SMPC hits to that of the native peptide, the binding affinity of the small molecule fragment is estimated. The best fragment binders will then be used as core scaffold to combinatorial libraries by replacing the rest of the peptide with small molecule functional groups. The combinatorial library generation approach of the present invention helps to grow fragment-sized molecules into lead-like molecules that can be quickly developed into potent inhibitors. The above concept is illustrated in FIG. 1

Screening and Hit Identification: In order to screen the SMPC library against a particular PPI target, a fluorescent polarization (FP) assay is set up where the displacement of the interacting peptide labeled with a fluorophore by the SMPCs can be observed and quantified appropriately. Comparison of the peptide control with any of the SMPC binders provides an estimate of the contribution of the binding of the small molecule fragment to the target. All the SMPC hits identified in the FP screening are further validated using biophysical screening techniques like Isothermal calorimetry (ITC).

Conversion of SMPC hit to a lead molecule: Once a fragment hit is identified there are multiple ways of converting the fragment to a lead molecule: A) Following traditional fragment growing/merging techniques, B) Utilizing high throughput amide chemistry and growing into the adjacent hotspots/pockets via combinatorial library generation. It is envisaged that the latter approach will be used for lead generation as it is higher throughput and it will take fraction of the time compared to the former technique. The disclosure is not limited to the above and includes alternate lead generation strategies.

In one aspect, the SMPCs are generated using amide chemistry. It is envisaged that the Smip platform described herein is applied to any protein-protein/peptide complex wherein there is availability of a protein-peptide complex and wherein the peptide binds in a linear or loop conformation to the host protein.

The platform technology described herein provides therapeutically relevant small molecule lead compounds which are further developed into drugs for various conditions, diseases or disorders.

In addition, the present technology is also useful for developing peptide diagnostic tools. Peptides are also routinely used in diagnostics. With the aid of phage display technology, we can identify novel and potent peptide binders against the antigen of interest. These peptides can then be converted into SMPCs using our approach. An SMPC library against a particular target has a huge diversity of small molecule fragments attached to the peptide. This diversity can enable specific identification of antigens. Along with this an SMPC hit may be more potent and specific than the phage display peptide and hence can act as a more sensitive and specific diagnostic tool.

In yet another aspect, the innovative SMPC approach disclosed herein is useful in developing new peptide based vaccines or improving existing ones. The addition of a small molecule moiety may have the following benefits to peptide based vaccines
a) Improved physicochemical properties
b) Better cell permeability
c) More potent and specific interaction

The present invention is advantageous over existing drug screening technologies.

In one embodiment the invention allows exploitation of structural knowledge and PPI hot spots.

In another embodiment the SMPC libraries are target specific and tailor-made resulting in less false positives and false negatives as compared to other existing routine techniques, for example, High Throughput Screening employing classical fragment based screening techniques such as SPR, NMR result in a lot of false positive and negative hits.

The technology also provides a platform for generating peptide-based vaccines by attaching small molecules to target peptide using amide coupling which is described in the instant specification.

In yet another embodiment, the present technology avoids the need for co-crystal structures of fragments which is otherwise necessary in FBDD techniques.

In a most preferred embodiment the said platform technology is faster and cheaper than existing screening techniques such as FBDD.

Further advantages of the present invention can be listed as under:
Reduced time frame (and thus effort) from fragment to lead generation as compared to traditional fragment based screening techniques while retaining non-peptidomimetic features;
The invention provides generation of a small molecule-peptide microarray wherein the microarray is defined as a minimum of two and a maximum of a billion small molecule-peptide conjugates.
the technology guarantees finding hits against challenging PPI targets; and
the follow up chemistry is simple.

FIG. 2 compares the Smip platform and the traditional fragment based screening techniques highlighting the difference in time needed to get to the small molecule lead.

Table 1 below highlights the benefits and economics of the Smip Platform technology

TABLE 1

| | | Cost of | Lead | |
|---|---|---|---|---|
| | | Screening | Generation | |
| | Target | per 100 | Time | Drug |
| Technology | Specificity | cmpds.($) | (Months) | Likeness |
| Smip | YES | 20,000 | 12 | GOOD |
| FBDD | NO | <100,000 | 18-48 | GOOD |
| HTS | NO | <20,000 | 6-12 | POOR |

Furthermore, besides traditional HTS and FBDD, some of the other competing technologies in the PPI hit discovery field are macrocycles, stapled peptides, and peptidomimetics. Macrocycles technology (e.g., Encycle Therapeutics and Bicycle Therapeutics) only target PPI complexes where the substrate peptides adopt a loop/curved conformation in a manner that can be exploited in designing these cyclic peptides. Stapled peptides/helices technology (e.g., Aileron Therapeutics) can only be applied to substrate peptides that have a helical conformation. Compared to both the methods above, our technology can be applied to any substrate peptide conformation (e.g., loop, helix, sheet, random), making our approach more powerful than our competitors. While the peptidomimetic approach enables quick development of peptide drugs, these molecules have been shown to be impermeable in cells with poor physicochemical properties that may result in failures in clinics. There are some prior publications detailing the design of small molecule peptide hybrids (He et al., Bioroganic & Medicinal Chemistry, 21: 7539-7548, 2013). These authors describe the generation of these hybrids using a variety of chemical methods. However, none of them use an amide bond to create these conjugates and none of them report using a high throughput library generation format. The technology of the present invention and the expertise developed will allow the applicants to outpace available technologies in terms of target selection, chemical diversity, hit identification, and lead development with respect to both time and cost.

The technology of the present invention is unique, innovative and advantageous compared to existing screening technologies as it enables us to:

a) engage the PPI target to expose relevant binding pockets

b) exploit any peptide conformation (linear, secondary, or tertiary)

c) explore wider chemical space

d) discover small molecule lead compounds cheaper and faster with efficient medicinal chemistry

e) select specific PPI targets for drug design

f) replace biologics with small molecules

The Examples provided herein are for illustrative purposes only and not to be construed as limiting the scope of the invention.

### EXAMPLES

Two emerging epigenetic PPI targets of therapeutic interest that had previously been unsuccessfully screened using classic high throughput screening (HTS) were picked to test the novel approach and identify novel SMPC hits and convert them into efficacious non-peptidic small molecule inhibitors of these two targets.

### Example 1

Staphylococcal nuclease like domain (SND1) SND1 is a ubiquitously expressed multifunctional protein involved in transcriptional regulation, RNA splicing and RNA metabolism. Recently, multiple publications highlighted its role in benign prostate cancer, hepatocellular carcinoma and colon cancer (Emdad et al., Neuro Incology, 17:419-429, 2015; Ma et al., Oncotarget, 6: 17404-17416, 2015; Zagryazhskaya et al., Oncotarget, 6: 12156-121573, 2015). Routine HTS screening in laboratories around the world has not resulted in any validated small molecule hits for drug design and development.

Work Step 1: SMPC Design, Synthesis and Screening

The co-crystal structure of SND1 with a heptapeptide fragment of its binding partner RIWI protein was used. The design involved the replacement of the N-terminal Arginine-Alanine peptide fragment with a collection of commercially available fragment building blocks with free carboxylates. In Silico coupling of the fragments to the remainder of the hexapeptide sequence was carried out and then these SMPCs were docked into the SND1 peptide-binding pocket. From the In Silico data, a library of 30 SMPCs against SND1 were selected and synthesized. The SMPC synthesis was carried out using standard solid phase manual peptide synthesis.

Work Step 2: Small Molecule Lead Convesrion

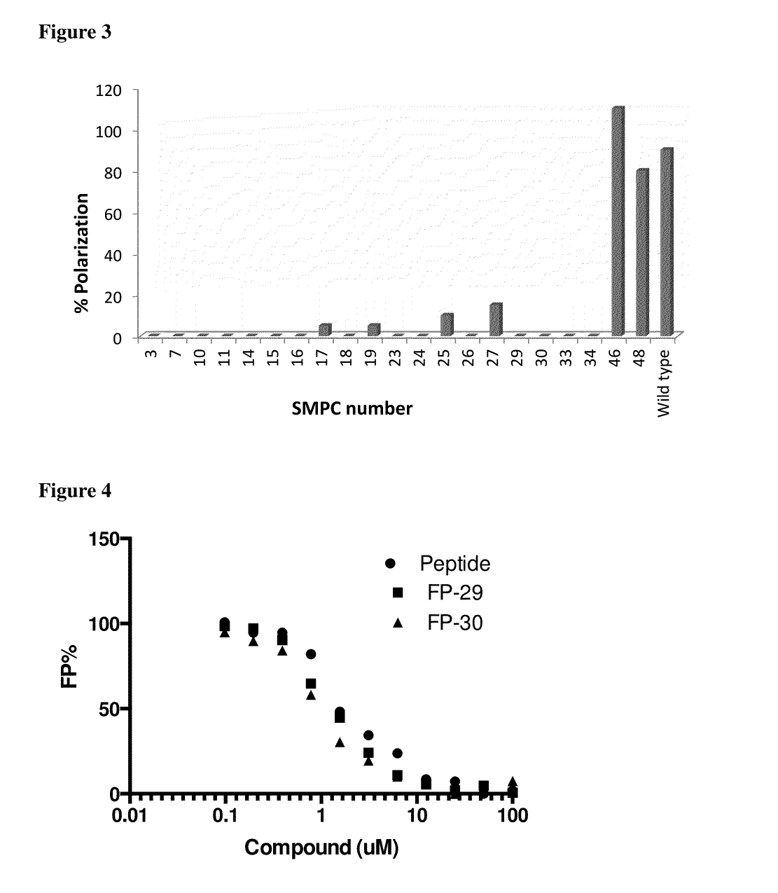

A library of 20 SMPCs were synthesized against SND1 (TUDOR). FIG. 3 shows that SMPC-46 (FP-29 in the figure) and SMPC-48 (FP-30 in the figure) elicited good fluorescent polarization responses as compared to the peptide substrate. FIG. 4 and Table 2 show the dose responses indicating that SMPC-46 binds better than the peptide substrate.

TABLE 2

| Substrate | K_{d}(μM) |
|---|---|
| Peptide Substrate | 10 |
| SMPC-46 | 5 |
| SMPC-48 | 28 |

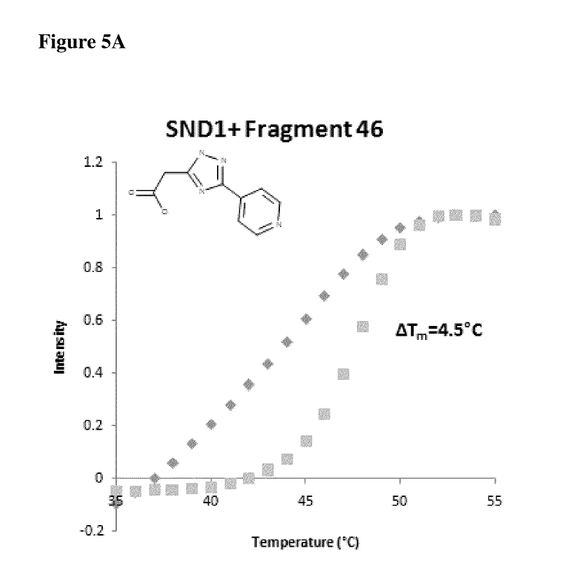

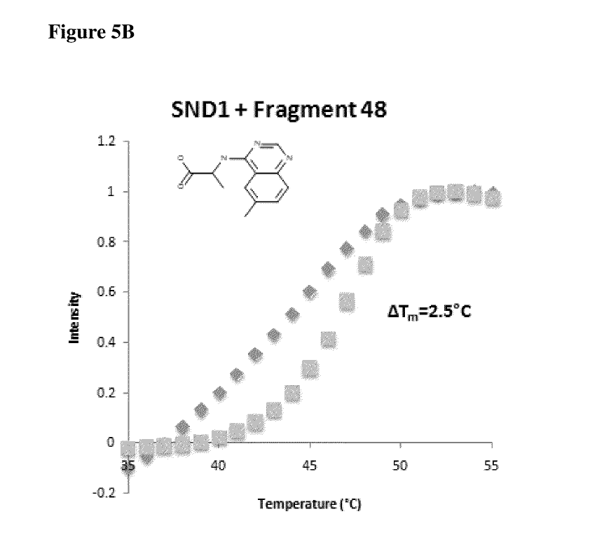

Fragments when tested by themselves showed binding to SND1 by thermal shift and ITC (FIG. 5A and 5B). Table 3 shows the results of thermal shift and ITC for both Fragment 46 and Fragment 48 in comparison to peptide substrate.

TABLE 3

| | K_{d} ITC | ΔTₘ*(Thermal | |
|---|---|---|---|
| Substrate | (μM) | shift)° C. | STD NMR |
| Peptide Substrate | 6 | 5.0 | Strong Signal |
| Fragment-46 | 50 | 4.5 | Strong Signal |
| Fragment-48 | N.D. | 2.5 | Weak Signal |

These result show that the SMPC (Smip) platform technology of the present invention can identify hits to SND1 which is a tough target, where other in-house compound library screening have proved unsuccessful.

### Example 2

WD Repeat Containing Protein 5 (WDR5) WDR5 is an epigenetic histone reader protein, which is therapeutically relevant drug target in Acute Myeloid Leukemia (AML). WDR5 is an integral part of the PRC2 complex (Senisterra et al., The Biochemical Journal, 449: 151-159, 2013), composed of 5 proteins. The integrity of the PRC complex is essential for methyl transferase activity of MLL1, a critical protein in AML. Very recently WDR5 was shown to interact with MLL1 protein directly leading to the therapeutic hypothesis that disruption of this complex will result in apoptosis in AML cells. The Applicant has used the approach disclosed in the present specification to illustrate that it is possible to identify novel small molecule inhibitor to disrupt the WDR5-MLL complex in vitro and in vivo in a resource efficient manner.

Work Step 1: SMPC Design, Synthesis and Screening

This study involved the design and synthesis of only 20 SMPCs, with a pentapeptide fragment of its binding partner MLL1 protein. Our design involved the replacement of the N-terminal arginine of the WIN peptide with a collection of commercially available fragment building blocks with free carboxylates. In Silico coupling of the fragments to the remainder of the tetrapeptide sequence was carried out and the SMPCs were docked into the WDR5 peptide-binding pocket.

The SMPCs when screened in a FP assay resulted in one confirmed hit (PRX-0065) (hit rate of 5%) with a Kd of 0.85 μM when compared to the substrate peptide Kd of 1.3 as shown in Table 4.

TABLE 4

| Substrate | K_{d}(μM) |
|---|---|
| Peptide Substrate | 1.3 |
| PRX-0065 | 0.85 |
| (SMPC Hit) | |

Work Step 2: Small Molecule Lead Conversion

SMPC hit (PRX-0065) against WDR5 for small molecule lead conversion was selected. By replacing the tri-peptide part of the SMPC with a library of commercially available building blocks, we were able to design a virtual library of 7000 small molecules. Docking of the small molecule set was carried out using a proprietary algorithm against WDR5, and top 100 hits were selected for synthesis. Eighty Four s small molecules were synthesized in just 6 weeks due to the ease of chemical synthesis.

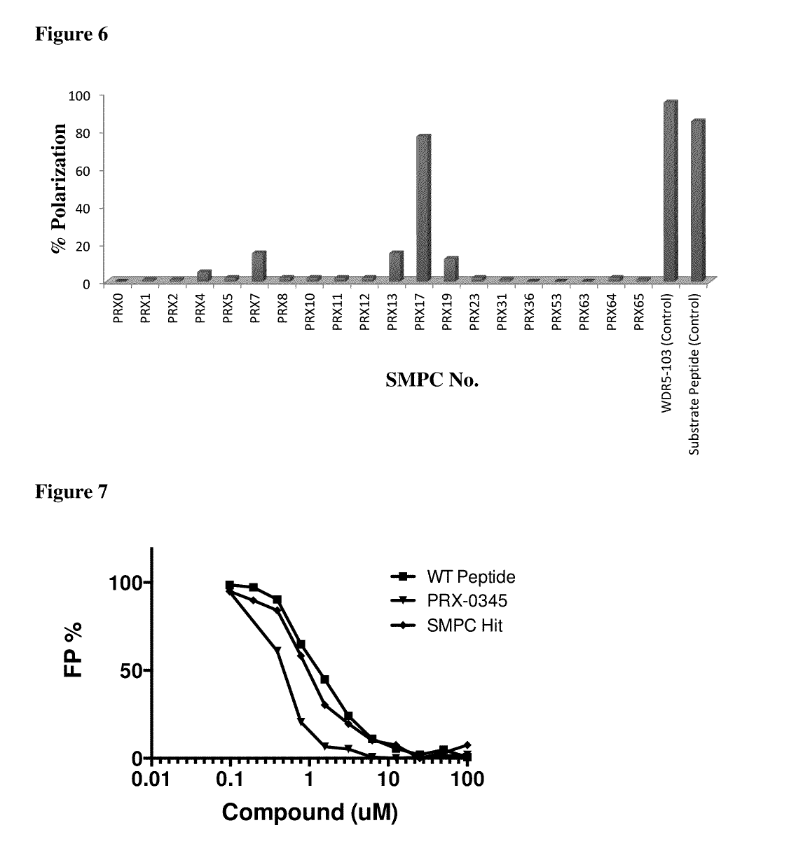

When screened in the biochemical assays (Fluorescent polarization and activity), two small molecule leads with similar structures (PRX-0345 and PRX-0365) respectively showed 0.45 and 2 μM potency (FIG. 6). PRX-0345 is a rule of 5 complaint small molecule with drug like features and it showed efficient toxicity at two different compound concentrations of 5 and 10 μM respectively (FIG. 7).

TABLE 5

| | K_{d} ITC | ΔTₘ*(Thermal |
|---|---|---|
| Substrate | (μM) | shift)° C. |
| Peptide Substrate | 1.3 | 2.0 |
| PRX-0065 | 0.9 | 0.85 |
| (SMPC Hit) | | |
| PRX-0345 (Small | 0.49 | 0.4 |
| Molecule Lead) | | |

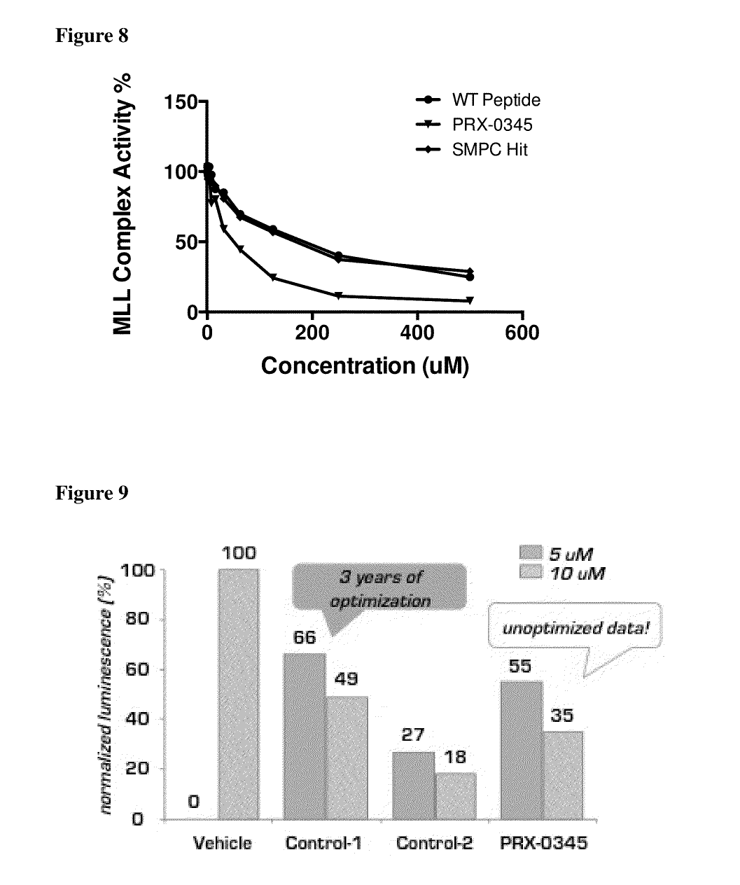

FIG. 8 provides dose response MLL complex activity comparing substrate peptide PRX-0345 and the SMPC hit PRX-0065.

### Example 3

Cell Based Response

A patient derived leukemia cell line was transformed with MLL-AF9 fusion vector. PRX-0345 and the control compounds, OICR-9429, and MM-102 were transfected into the cells at 5 and 10 μM. The results showed that when screened at the lowest compound concentration of 5 μM, PRX-0345 was able to kill MLL-AF9 leukemia cells more efficiently than OICR-9429 (FIG. 9).

Overall the studies described herein clearly demonstrate the identification of SMPC hits against two challenging PPI targets and the efficient conversion of one SMPC hit into a cell active small molecule lead that can be further developed for small molecule indicating the utility and operability of the invention described.

We claim:

1) A method of developing small molecule lead compounds comprising generating a library of small molecule-peptide conjugates (SMPCs) by in silico highthroughput screening or highthroughput coupling of small molecule fragments to a peptide.

2) The method of claim 1 wherein the small molecule fragment is attached to the peptide via an amide bond.

3) A method of generating a library of SMPCs comprising in silico highthroughput coupling of small molecule fragments to a peptide via an amide bond.

4) The method of claim 1 or 3 wherein the SMPCs are synthesized on a solid surface.

5) The method of claim 4 wherein the solid surface is any solid membrane.

6) The method of claim 4 wherein the solid surface is selected from a group consisting of cellulose, gold and glass slide.

7) The method of claim 1 or 3 wherein the SMPCs are synthesized using highthroughput peptide microarrays.

8) A method for generating a small molecule-peptide conjugate microarray wherein the microarray ranges from a minimum of two and a maximum of a billion small molecule peptide conjugates.

9) A method of developing small molecule lead compounds comprising the steps of:
a) in silico high throughput screening of small molecule fragments against protein-peptide or protein-protein target;
b) generating a library of SMPCs by attaching the small molecule fragments to the peptide via an amide bond;
c) selecting and synthesizing the SMPC hits;
d) screening the SMPCs against a target of interest to identify the small molecule fragment hits; and
e) conversion of the small molecule fragment to small molecule lead compounds for a corresponding target.

10) A method of generating a high throughput library of SMPCs comprising synthesizing highthroughput peptide microarrays on a solid surface by coupling small molecule building blocks to the peptide.

11) The method of claim 10 wherein the coupling of small molecule building blocks onto the peptide is via amide coupling.

12) The method of claim 10 wherein the coupling of small molecule to the peptide is done while immobilized on a solid surface.

13) The method of claim 10 wherein the coupling of small molecule to the solid surface is by using a linker selected from the group consisting of biotin, PEG and any solid small molecule linker.

14) A method of use of SMPC peptide microarrays for biochemical and biophysical assays.

15) A composition comprising the SMPC synthesized by the method of claim 1.

16) A method of use of SMPC attached to gold surface in Surface Plasmon Resonance.

17) A method of use of SMPC in generating peptide-based vaccines comprising attaching small molecules to target peptide using amide coupling.

18) A method of use of SMPC as a diagnostic tool.
